(19) United States
(12) Patent Application Publication
(10) Pub. No.: US 2007/0032458 A1
(43) Pub. Date: Feb. 8, 2007

(54) Compound having activity for inhibiting dioxin toxicity, pharmaceutical composition comprising the compound and method for treating disease resulting from dioxin toxicity using the compound

(75) Inventors: Pann Ghill Suh, Pohang-city (KR); Sung Ho Ryu, Pohang-city (KR); Sun Hee Kim, Seogwipo-city (KR); Yun Hee Kim, Pohang-city (KR)

Correspondence Address: ROTHWELL, FIGG, ERNST & MANBECK, P.C., 1425 K STREET, N.W., SUITE 800, WASHINGTON, DC, 20005, US

(73) Assignees: POSTECH FOUNDATION, Pohang-city (KR); POSTECH ACADEMY-INDUSTRY FOUNDATION, Pohang-city (KR)

(21) Appl. No.: 11/476,215

(22) Filed: Jun. 28, 2006

Related U.S. Application Data
(60) Provisional application No. 60/694,256, filed on Jun. 28, 2005.

(30) Foreign Application Priority Data
Jun. 9, 2006 (KR) 10-2006-0051989

(51) Int. Cl. A61K 31/655; C07C 245/02

(52) U.S. Cl. 514/150; 534/875; 534/792

(57) ABSTRACT

Provided are a compound having an activity for inhibiting dioxin toxicity, a pharmaceutical composition including the compound and a method of treating disease resulting from dioxin toxicity using the compound. More particularly, the compound is represented by formula 1: 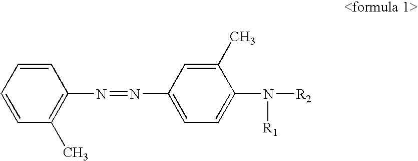
where, R₁ may be a hydrogen atom, a C1-C5 alkyl group or an alkylketone group; and R₂ may be a hydrogen atom, a C1-C10 alkyl group or —COR₃, wherein R3 may be a C1-C5 alkyl group, a C1-C5 alkylamide group, a C3-C12 aryl group or a heteroaryl group. A compound having an activity for inhibiting dioxin toxicity by directly antagonizing a dioxin receptor and specifically lessening dioxin-associated toxicity without an adverse effect resulting from an overdose of the compound or an outbreak of another disease can be obtained.

### CROSS-REFERENCE TO RELATED PATENT APPLICATION

This application claims the benefit of U.S. Provisional Application No. 60/694,256 filed on Jun. 28, 2005, in the United States Patent and Trademark Office and Korean Patent Application No. 10-2006-0051989, filed on Jun. 9, 2006, in the Korean Intellectual Property Office, the disclosures of which are incorporated herein in their entirety by reference.

### BACKGROUND OF THE INVENTION

1. Field of the Invention

The present invention relates to a compound having an activity for inhibiting dioxin toxicity, a pharmaceutical composition including the compound and a method for treating disease resulting from dioxin toxicity using the compound, and more particularly, to a compound having an activity for inhibiting dioxin toxicity by directly antagonizing a dioxin receptor and specifically lessening dioxin-associated toxicity without an adverse effect resulting from an overdose of the compound or an outbreak of another disease, a pharmaceutical composition including the compound, and a method of treating disease resulting from dioxin toxicity using the compound.

2. Description of the Related Art

2,3,7,8-tetrachlorodibenzo-p-dioxin (TCDD, hereinafter referred to as dioxin) is a highly persistent environmental contaminant and one of the most potent man-made toxicants. Adverse effects of dioxin include a number of phenomena such as lethality (Blankenship et al., 2003), tumor promotion (Bertazzi et al., 1999; Cole et al, 2003), immunotoxicity (Kerkvliet, 1995; Ahmed et al., 2005), teratogenecity (Couture et al., 1990) and endocrine changes (Osteen and Sierra-River, 1997; Gregoraszczuk, 1995).

It is widely accepted that the majority of toxic effects of dioxin in higher organisms are mediated through activation of the cytosolic aryl hydrocarbon receptor (AhR) that is a ligand-activated transcription factor of the basic helix-loop-helix family (Mimura and Fujii-Kuriyama, 2003; Mandal, 2005). Upon binding a ligand such as dioxin, AhR translocates to the nucleus where it binds the aryl hydrocarbon receptor nuclear translocator (ARNT). This complex binds to enhancer sequences in target genes designated dioxin-responsive elements (DREs) that regulate expression of several genes including cytochrome P450 enzymes.

Cytochrome P450 enzymes catalyze the epoxidation of certain classes of xenobiotics. The consequence of this can be the generation of highly reactive electrophilic metabolites and hydroxyl radicals, which may ultimately lead to cancer (Shimada and Fujii-Kuriyama, 2003; Chen et al, 2004).

AhR activation caused by the binding of dioxin is assumed to be one of the first and key steps in the development of dioxin toxicity. Accordingly, the inhibition of AhR activation would be expected to provide protection against dioxin toxicity. More recently, chemicals found in foods such as flavone and resveratrol and their derivatives have been reported to exert antagonistic activity on the binding of dioxin to AhR (Amakura et al, 2003; Zhang et al, 2003). However, at high concentrations these compounds act as AhR agonists, causing an increase in cytochrome P450 expression. In addition, some reported AhR antagonists have a high affinity for the estrogen receptor (Suetsugi et al, 2003; de Medina et al, 2005), which may cause estrogen-related effects.

Therefore, there is a need for an effective method of treating diseases resulting from dioxin exposure by developing a compound that has antagonist activity on the binding of dioxin to AhR and does not act as an agonist.

### SUMMARY OF THE INVENTION

The present invention provides a compound having an activity for inhibiting dioxin toxicity by directly antagonizing a dioxin receptor and specifically lessening dioxin-associated toxicity without an adverse effect resulting from an overdose of the compound or an outbreak of another disease, a pharmaceutical composition including the compound, and a method for treating disease resulting from dioxin toxicity using the compound.

According to an aspect of the present invention, there is provided a compound having an activity for inhibiting dioxin toxicity and represented by formula 1 below:

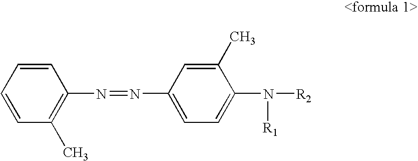

where, R₁ may be a hydrogen atom, a C1-C5 alkyl group or an alkylketone group; and

R₂ may be a hydrogen atom, a C1-C10 alkyl group or —COR₃, wherein R3 may be a C1-C5 alkyl group, a C1-C5 alkylamide group, a C3-C12 aryl group or a heteroaryl group.

According to another aspect of the present invention, there is provided a pharmaceutical composition including the compound at an effective amount for treatment.

According to another aspect of the present invention, there is provided a method for treating a disease resulting from dioxin toxicity, including administering an effective treatment amount of the compound to a patient having a disease resulting from dioxin toxicity.

### BRIEF DESCRIPTION OF THE DRAWINGS

The above and other features and advantages of the present invention will become more apparent by describing in detail exemplary embodiments thereof with reference to the attached drawings in which:

FIG. 1 illustrates Western blot analysis of cells treated with Compound 2 and flavone; and

FIG. 2 illustrates the ability in percentage of compounds of the present invention to inhibit the binding of ³[H]-dioxin to its receptor depending on the concentrations thereof.

### DETAILED DESCRIPTION OF THE INVENTION

Hereinafter, the present invention will now be described more fully with reference to the accompanying drawings, in which exemplary embodiments of the invention are shown. The invention may, however, be embodied in many different forms and should not be construed as being limited to the embodiments set forth herein; rather, these embodiments are provided so that this disclosure will be thorough and complete, and will fully convey the concept of the invention to those skilled in the art.

In an embodiment of the present invention, research on developing a compound inhibiting expression and an activity of enzymes that are specifically increased by dioxin has been performed. Dioxin toxicity is mediated through aryl hydrocarbon receptor (AhR). Upon binding dioxin, AhR translocates to the nucleus and increases a transcription of specific proteins including cytochrome P450 enzymes. Particularly, cytochrome P450 enzyme that is an enzyme mainly expressed in the liver is known to mediate a part of a toxicity resulting from dioxin, and dioxin causes a noticeable increase in cytochrome P450 enzyme expression. Accordingly, finding a compound inhibiting expression and activity of cytochrome P450 enzyme can be an effective indicator for finding a compound inhibiting dioxin toxicity.

According to an embodiment of the present invention, a compound having an activity for inhibiting dioxin toxicity and represented by formula 1 below is provided:

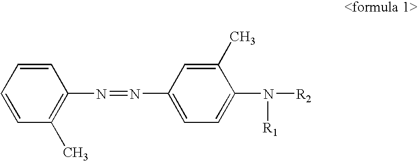

where, R₁ may be a hydrogen atom, a C1-C5 alkyl group or an alkylketone group; and

R₂ may be a hydrogen atom, a C1-C10 alkyl group or —COR₃, wherein R3 may be a C1-C5 alkyl group, a C1-C5 alkylamide group, a C3-C12 aryl group or a heteroaryl group.

The compound represented by formula 1 may be any one of compounds represented by formulae 2 through 8 below:

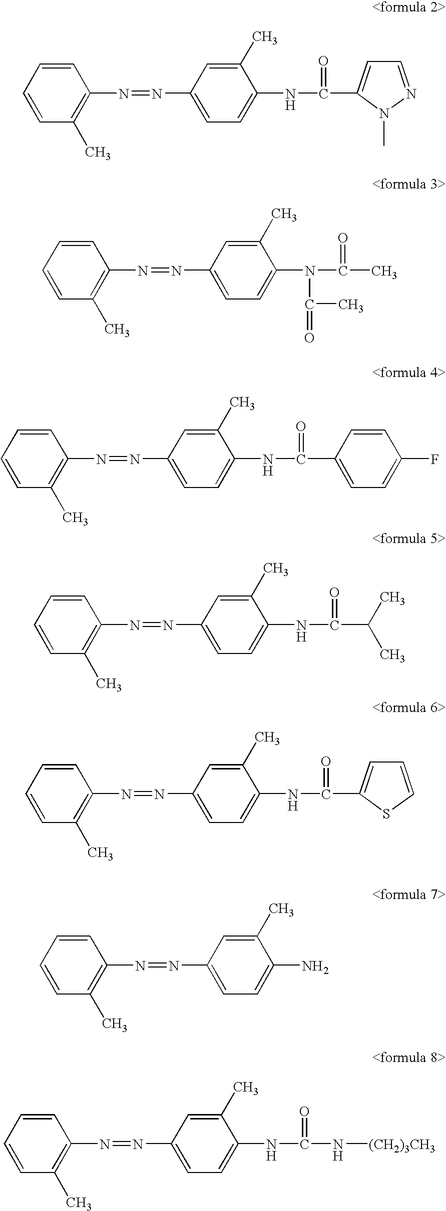

The compound may have an activity of inhibiting binding of dioxin to a dioxin receptor. Here, the dioxin receptor may be aryl hydrocarbon receptor (AhR). AhR activation caused by the binding of dioxin is one of the first and key steps in the development of dioxin toxicity. Accordingly, the compound represented by formula 1 inhibits binding dioxin to a dioxin receptor, inhibits a formation of a dioxin-AhR complex that is required to express cytochrome P450 enzyme, and thus inhibits expression of cytochrome P450 enzyme.

In addition, the compound represented by formula 1 also inhibits cytochrome P450 enzyme activity.

Therefore, the compound represented by formula 1 shows a great activity in treating a number of phenomena such as lethality, tumor promotion, immunotoxicity, teratogenecity and endocrine changes that are assumed to be mediated by dioxin.

In another embodiment of the present invention, there is provided a pharmaceutical composition including an effective treatment amount of the compound represented by formula 1.

The pharmaceutical composition may further include pharmaceutically acceptable additives. Examples of the additives may include at least one of excipients, disintegrants, binders, lubricants, and suspensions that are commonly used in the pharmaceutical field. For example, when the pharmaceutical composition is prepared in a solid formulation such as a tablet, a hard capsule, or the like, noncrystalline cellulose, lactose, low-substituted hydroxycellulose, and the like may be used as excipients; sodium starch glycolate, calcium hydrogen phosphate anhydride, and the like may be used as disintegrants; polyvinyl pyrrolidone, low-substituted hydroxypropylcellulose, hydroxypropylcellulose, and the like may be used as binders; magnesium stearate, silicon dioxide, talc, and the like may be used as lubricants; and a surfactant such as sorbitan esters or polysorbates that is commonly used in the pharmaceutical field may be used as suspensions.

The pharmaceutical composition according to the current embodiment of the present invention may be formulated as various forms including solid and liquid forms. Examples of the formulation may include a tablet, a capsule, a solution, a suspension, an emulsion and a syrup.

In another embodiment of the present invention, there is provided a method for treating a disease resulting from dioxin toxicity including administering an effective treatment amount of the compound represented by formula 1 to a patient having a disease resulting from the dioxin toxicity.

The compound may be administered to a patient in various forms such as an oral form and a parenteral form in a proper amount depending on the age, sex, and severity of disease of the patient. The administering amount may be easily regulated by those skilled in the clinical field of treating and preventing diseases. For example, the compound may be administered at a dose of 1 to 10 mg/kg a day.

Hereinafter, a more detailed description of the specific effect of the present invention will be provided with reference to the following Examples. The following Examples are for illustrative purposes only and are not intended to limit the spirit and scope of the invention.

Human Hepatocarcinoma Cell Culture

Hep G2 cell that is a human hepatocarcinoma cell line was cultured in an incubator with a culture medium of Dulbecco's Modified Eagle Medium (DMEM) containing 10% of Fetal Bovine Serum (FBS) at 37° C. under an atmosphere of 5% of CO₂.

Inhibition of Cytochrome P450 Enzyme Activities Using a Compound According to an Embodiment of the Present Invention

1×104 of the cultured human hepatocarcinoma cells were placed into wells of a 95 well plate. After 24 hours, the cells were treated with a solution in which compounds represented by formulae 2 through 8 (obtained from Chembridge Corporation, San Diego, Calif.) were dissolved in dimethyl sulfoxide (DMSO) in a concentration of 0.1%. After one hour, the cells were treated with 3 nM of 2,3,7,8-tetrachlorodibenzo-p-dioxin (obtained from Cambridge Isotope Laboratory, Andover, Mass., U.S.A.). Cytochrome P450 enzyme activity was measured after 24 hours and the measured activity was evaluated by measuring ethoxyresorufin-O-deethylase activity (EROD, Ciolino et al, 1998). The results are shown in Table 1.

TABLE 1

| | Dioxin-induced EROD activity |
|---|---|
| Compound | inhibition IC₅₀ (nM) |
| Compound 2 | 30 ± 2 |
| Compound 3 | 75 ± 4 |
| Compound 4 | 105 ± 11 |
| Compound 5 | 110 ± 7 |
| Compound 6 | 153 ± 10 |
| Compound 7 | 215 ± 42 |
| Compound 8 | 450 ± 75 |

Decrease in expressed amount of cytochrome P450 treated with Compound 2 according to an embodiment of the present invention

3×10⁵ of the cultured human hepatocarcinoma cells were placed into tissue culture plates and cultured for 24 hours. After 24 hours, the cells were treated with a solution in which Compound 2 (obtained from Chembridge Corporation, San Diego, Calif.) was dissolved in dimethyl sulfoxide (DMSO) in a concentration of 0.1%. After one hour, the cells were treated with 3 nM of 2,3,7,8-tetrachlorodibenzo-p-dioxin (obtained from Cambridge Isotope Laboratory, Andover, Mass., U.S.A.). After 24 hours, the culture medium was removed by suction and the cells were washed with phosphate buffer solution. Then the cells were lysed and the protein was quantified. As a result of Western blot analysis using anti-cytochrome P450 1A1 antibody (first antibody) to measure the amount of the expressed cytochrome P450 1A1, it was identified that Compound 2 inhibited expression of cytochrome P450 1A1 that is increased by dioxin.

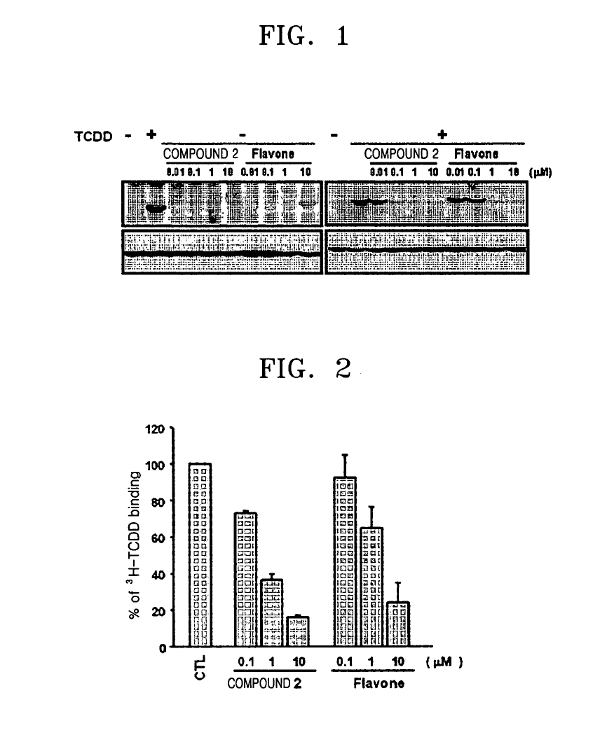

In FIG. 1, flavone that has been widely reported to inhibit dioxin toxicity even though it increases dioxin toxicity at high concentrations was used as a positive control group. FIG. 1 illustrates Western blot analysis of cells treated with Compound 2 and flavone.

As illustrated in FIG. 1, Compound 2 has an excellent effect in decreasing the amount of expressed cytochrome P450 enzyme at various concentrations.

Inhibition of Binding Dioxin to a Dioxin Receptor Using a Compound According to an Embodiment of the Present Invention

Compound 2 and 3[H]-dioxin were added to cytoplasm of hepatocarcinoma cells and analysis on binding of ligand-receptor was performed using hydroxylapatite. As a result, it was confirmed that Compound 2 inhibited the binding of 3[H]-dioxin to its receptor. The results are illustrated in FIG. 2.

FIG. 2 illustrates the ability in percentage of Compound 2 to inhibit the binding of 3[H]-dioxin to its receptor. It was confirmed that the binding ability of 3[H]-dioxin to its receptor decreased as the concentration of Compound 2 increased from 0.1 to 1 and to 10 μM.

According to the present invention, a compound having an activity for inhibiting dioxin toxicity by directly antagonizing a dioxin receptor and specifically lessening dioxin-associated toxicity without an adverse effect resulting from an overdose of the compound or an outbreak of another disease can be obtained.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A compound having an activity for inhibiting dioxin toxicity and represented by formula 1 below: 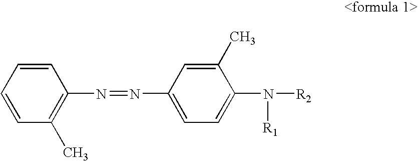
where, R₁ is one selected from the group consisting of a hydrogen atom, a C1-C5 alkyl group and an alkylketone group; and
R₂ is one selected from the group consisting of a hydrogen atom, a C1-C10 alkyl group and —COR₃, wherein R3 is one selected from the group consisting of a C1-C5 alkyl group, a C1-C5 alkylamide group, a C3-C12 aryl group and a heteroaryl group.

2. The compound of claim 1, wherein the compound is any one of compounds represented by formulae 2 through 8 below:

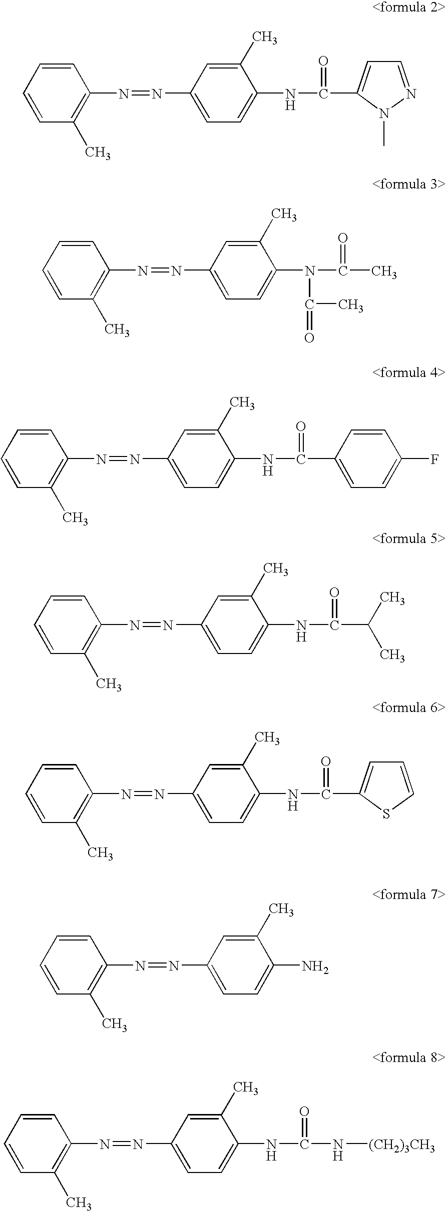

3. The compound of claim 1, wherein the compound inhibits binding of dioxin to a dioxin receptor.

4. The compound of claim 3, wherein the dioxin receptor is aryl hydrocarbon receptor (AhR).

5. The compound of claim 3, wherein the compound inhibits expression of cytochrome P450 enzyme by inhibiting the binding of dioxin to the dioxin receptor.

6. The compound of claim 1, wherein the compound inhibits cytochrome P450 enzyme activity.

7. The compound of claim 1, wherein the compound has an activity of treating lethality, tumor promotion, immunotoxicity, teratogenecity, and endocrine changes.

8. A pharmaceutical composition comprising the compound of claim 1 at an effective amount for treatment.

9. The pharmaceutical composition of claim 8, further comprising pharmaceutically acceptable additives.

10. The pharmaceutical composition of claim 8, wherein the composition is in a form selected from the group consisting of a tablet, a capsule, a solution, a suspension, an emulsion and a syrup.

11. A method for treating a disease resulting from dioxin toxicity, comprising administering an effective treatment amount of the compound of claim 1 to a patient having a disease resulting from dioxin toxicity.

12. The method of claim 11, wherein the compound is administered at a dose of 1 to 10 mg/kg a day.

13. The method of claim 11, wherein the disease comprises at least one disease selected from the group consisting of lethality, tumor promotion, immunotoxicity, teratogenecity and endocrine changes.
